(19) United States
(12) Patent Application Publication
(10) Pub. No.: US 2009/0192344 A1
(43) Pub. Date: Jul. 30, 2009

(54) SURGICAL DEVICES FOR MANIPULATING TISSUE

(75) Inventors: Gregory J. Bakos, Mason, OH (US); Christie Mrie Cunningham, Cincinnati, OH (US); Kendall Lee Dobler, Loveland, OH (US); William D. Fox, New Richmond, OH (US); Christopher J. Hess, Cincinnati, OH (US); Gary L. Long, Cincinnati, OH (US); Rudolph Henry Nobis, Mason, OH (US); Carl J. Shurtleff, Mason, OH (US); James T. Spivey, Cincinnati, OH (US); David Stefanchik, Morrow, OH (US); Michael J. Stokes, Cincinnati, OH (US); Omar J. Vakharia, Cincinnati, OH (US); James W. Voegele, Cincinnati, OH (US)

Correspondence Address: K&L GATES LLP, 535 SMITHFIELD STREET, PITTSBURGH, PA, 15222, US

(73) Assignee: Ethicon Endo-Surgery, Inc., Cincinnati, OH (US)

(21) Appl. No.: 12/019,461

(22) Filed: Jan. 24, 2008

(51) Int. Cl. A61N 2/10; A61N 2/00

(52) U.S. Cl. 600/12; 600/9

(57) ABSTRACT

A surgical kit can be used to manipulate tissue within the body of a patient to create a working space within the body to allow a surgeon to easily access and work within the body using various surgical instruments. A surgical kit can include an implant comprised of a magnetic material which can be engaged with tissue within the body. The kit can further include a surgical instrument having a magnet which can be used to manipulate the implant and tissue engaged therewith. A surgical kit can include an anchor and a hanger configured to engage tissue at different locations within the body and a connection member engaged with the anchor and the hanger such that the connection member can be pulled to move the anchor toward the hanger. A surgical instrument which utilizes a vacuum provided to one or more movable members can also be used to manipulate tissue.

### BACKGROUND

i. Field of the Invention

The present invention generally relates to surgical devices and, more particularly, to surgical devices for manipulating tissue within a patient's body.

ii. Description of the Related Art

Traditional, or open, surgical techniques may require a surgeon to make large incisions in a patient's body in order to access a tissue treatment region, or surgical site. In some instances, these large incisions may prolong the recovery time of and/or increase the scarring to the patient. As a result, minimally invasive surgical techniques are becoming more preferred among surgeons and patients owing to the reduced size of the incisions required for various procedures. In some circumstances, minimally invasive surgical techniques may reduce the possibility that the patient will suffer undesirable post-surgical conditions, such as scarring and/or infections, for example. Further, such minimally invasive techniques can allow the patient to recover more rapidly as compared to traditional surgical procedures.

Endoscopy is one minimally invasive surgical technique which allows a surgeon to view and evaluate a surgical site by inserting at least one cannula, or trocar, into the patient's body through a natural opening in the body and/or through a relatively small incision. In use, an endoscope can be inserted into, or through, the trocar so that the surgeon can observe the surgical site. In various embodiments, the endoscope may include a flexible or rigid shaft, a camera and/or other suitable optical device, and a handle portion. In at least one embodiment, the optical device can be located on a first, or distal, end of the shaft and the handle portion can be located on a second, or proximal, end of the shaft. In various embodiments, the endoscope may also be configured to assist a surgeon in taking biopsies, retrieving foreign objects, and introducing surgical instruments into the surgical site.

Laparoscopic surgery is another minimally invasive surgical technique where procedures in the abdominal or pelvic cavities can be performed through small incisions in the patient's body. A key element of laparoscopic surgery is the use of a laparoscope which typically includes a telescopic lens system that can be connected to a video camera. In various embodiments, a laparoscope can further include a fiber optic system connected to a halogen or xenon light source, for example, in order to illuminate the surgical site. In various laparoscopic, and/or endoscopic, surgical procedures, a body cavity of a patient, such as the abdominal cavity, for example, can be insufflated with carbon dioxide gas, for example, in order to create a temporary working space for the surgeon. In such procedures, a cavity wall can be elevated above the organs within the cavity by the carbon dioxide gas. Carbon dioxide gas is usually used for insufflation because it can be easily absorbed and removed by the body.

In at least one minimally invasive surgical procedure, an endoscope and/or laparoscope can be inserted through a natural opening of a patient to allow a surgeon to access a surgical site. Such procedures are generally referred to as Nature Orifice Transluminal Endoscopic Surgery or (NOTES)™ and can be utilized to treat tissue while reducing the number of incisions, and external scars, to a patient's body. In various NOTES procedures, for example, an endoscope can include at least one working channel defined therein which can be used to allow the surgeon to insert a surgical instrument therethrough in order to access the surgical site.

### SUMMARY

In at least one form of the invention, surgical devices can be utilized to manipulate tissue within the body of a patient to create a working space within the body. In various embodiments, the working space can allow a surgeon to easily access a surgical site using various surgical instruments. In at least one embodiment, a surgical device kit can include an anchor, a connection member, and a hanger, wherein the anchor can be configured to engage tissue at a first location within the body, such as an organ, for example, and wherein the hanger can be configured to be engaged with tissue at a second location within the body, such as a cavity wall, for example. In various embodiments, the connection member can be operably associated with the anchor and the hanger such that the tissue engaged with the anchor can be moved relative to the tissue engaged with the hanger. In at least one embodiment, a force can be applied to the connection member to pull the anchor toward the hanger. In various embodiments, the hanger, anchor, and/or connection member can be inserted into the body through a cannula and/or trocar, for example, which can be positioned within an incision and/or natural opening in the patient's body.

In at least one form of the invention, a surgical device kit can include at least one magnetic implant and, in addition, a surgical instrument having at least one magnet, such as a permanent magnet and/or electromagnet, for example. In at least one embodiment, the magnetic implant can be at least partially comprised of iron and, in various embodiments, the implant can be inserted into a surgical site through a cannula and/or trocar, for example. In various embodiments, the magnetic implant, or implants, can be attached to, or otherwise engaged with, tissue located within and/or proximate to the surgical site. In at least one embodiment, the surgical instrument can be positioned relative to the body and can emit at least one magnetic field configured to motivate the magnetic implants, and the tissue engaged therewith. In various embodiments, the magnets of the surgical instrument can be positioned external to the patient's body wherein the magnets can be configured to move the implants and tissue associated therewith to create working space around the surgical site.

In at least one form of the invention, a surgical instrument can be configured to apply a negative fluid pressure, or suction, to tissue within and/or surrounding a surgical site. In various embodiments, the surgical instrument can include at least one movable member which can engage the tissue via the suction and manipulate the tissue in order to create working space within the surgical site. In at least one embodiment, the instrument can include a first member configured to receive an endoscope, wherein the first member can include a channel in fluid communication with a vacuum source. The instrument can further include a tissue-contacting surface having an aperture therein which is in fluid communication with the channel. In various embodiments, the surgical instrument can further include a second member which can be movable relative to the first member, wherein the second member can also include a channel in fluid communication with a vacuum source, a tissue-contacting surface, and at least one aperture in the tissue-contacting surface which is in fluid communication with the vacuum source. In use, vacuum can be supplied to the first member and the second member such that they can hold tissue relative thereto and, when, the second member is moved relative to the first member, the tissue associated with the second member can be moved relative to the tissue associated with the first member.

### BRIEF DESCRIPTION OF THE FIGURES

The above-mentioned and other features and advantages of this invention, and the manner of attaining them, will become more apparent and the invention itself will be better understood by reference to the following description of embodiments of the invention taken in conjunction with the accompanying drawings, wherein:

FIG. 1 is a diagram of surgical devices being used to manipulate soft tissue, the devices including an anchor, a connection member, a hanger, and an endoscope in accordance with one non-limiting embodiment of the present invention;

FIG. 2 is another diagram of surgical devices being used to manipulate soft tissue, the devices including an anchor, a connection member, and a hanger in accordance with one non-limiting embodiment of the present invention;

FIG. 3 is a diagram of surgical devices including a needle and a flexible wire in accordance with one non-limiting embodiment of the present invention;

FIG. 4 is a diagram of the needle of FIG. 3 inserted through a cavity wall of a patient's body;

FIG. 5 is a diagram of the needle of FIG. 3 after it has been re-inserted through the cavity wall to create a loop within the patient's body;

FIG. 6 is a diagram of an endoscope inserted through the loop of FIG. 5;

FIG. 7 is a diagram of the wire of FIG. 3 having end portions tied together to retain the endoscope relative to the cavity wall;

FIG. 8 is a diagram of surgical devices including a hanger configured to hang from a cavity wall of a patient's body wherein the hanger is configured to hold an endoscope in position in accordance with one non-limiting embodiment of the present invention;

FIG. 9 is a perspective view of a surgical device including a connection member, a first attachment member, and a second attachment member in accordance with one non-limiting embodiment of the present invention;

FIG. 10 is a diagram of the surgical device of FIG. 9 illustrating the first attachment member engaged with a cavity wall of a patient's body and the second attachment member engaged with an organ in the surgical site in accordance with one non-limiting embodiment of the present invention;

FIG. 11 is a diagram of a surgical device in accordance with one non-limiting embodiment of the present invention which is configured to manipulate tissue;

FIG. 12 is side view of a surgical device including an overtube, a tissue-contacting member extending from the overtube, and an instrument director in accordance with one non-limiting embodiment of the present invention;

FIG. 13 is a side view of a surgical device including an overtube and a tissue-contacting member in accordance with one non-limiting embodiment of the present invention;

FIG. 14 is a partial cut away perspective view of a surgical device including an overtube and a movable member in accordance with one non-limiting embodiment of the present invention;

FIG. 15 is a side view of the movable member of the surgical device of FIG. 14;

FIG. 16 is a cross-sectional view of the surgical device of FIG. 14 wherein the surgical device is being used in conjunction with an endoscope and another surgical instrument in accordance with one non-limiting embodiment of the present invention;

FIG. 17 is a perspective view of the device of FIG. 14 illustrating a manifold positioned on an end of the overtube in accordance with one non-limiting embodiment of the present invention;

FIG. 18 is a side view of a surgical device including two movable jaw members in fluid communication with a vacuum manifold in accordance with one non-limiting embodiment of the present invention;

FIG. 19 is a perspective view of the surgical device of FIG. 18 illustrating a surgical instrument extending through a passageway defined by the manifold;

FIG. 20 is a diagram of a surgical device kit configured to manipulate an organ within a surgical site in accordance with one non-limiting embodiment of the present invention;

FIG. 21 is a diagram of a surgical device kit including a magnetic implant attached to an organ and, in addition, a magnetic member configured to motivate the magnetic implant where the magnetic member is positioned relative to a cavity wall in accordance with one non-limiting embodiment of the present invention;

FIG. 22 is a diagram of a surgical device kit including a magnetic member and, in addition, a plurality of magnetic implants attached to an organ in accordance with one non-limiting embodiment of the present invention;

FIG. 23 is a diagram of another surgical device kit including a magnetic member and, in addition, a plurality of magnetic implants positioned relative to an organ in accordance with one non-limiting embodiment of the present invention;

FIG. 24 is a diagram of a surgical device kit including a magnetic member positioned at the end of an elongate surgical instrument and, in addition, a pouch at least partially filled with a magnetic material in accordance with one non-limiting embodiment of the present invention;

FIG. 25 is a diagram of a surgical device kit including a magnetic member and a band comprising a magnetic material in accordance with one non-limiting embodiment of the present invention; and

FIG. 26 is a diagram of a surgical device kit including a plurality of magnetic implants attached to an organ and, in addition, a blanket having at least one magnetic member which is configured to be placed over a patient in accordance with one non-limiting embodiment of the present invention.

Corresponding reference characters indicate corresponding parts throughout the several views. The exemplifications set out herein illustrate preferred embodiments of the invention, in one form, and such exemplifications are not to be construed as limiting the scope of the invention in any manner.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the various embodiments of the present invention is defined solely by the claims. The features illustrated or described in connection with one exemplary embodiment may be combined with the features of other embodiments. Such modifications and variations are intended to be included within the scope of the present invention.

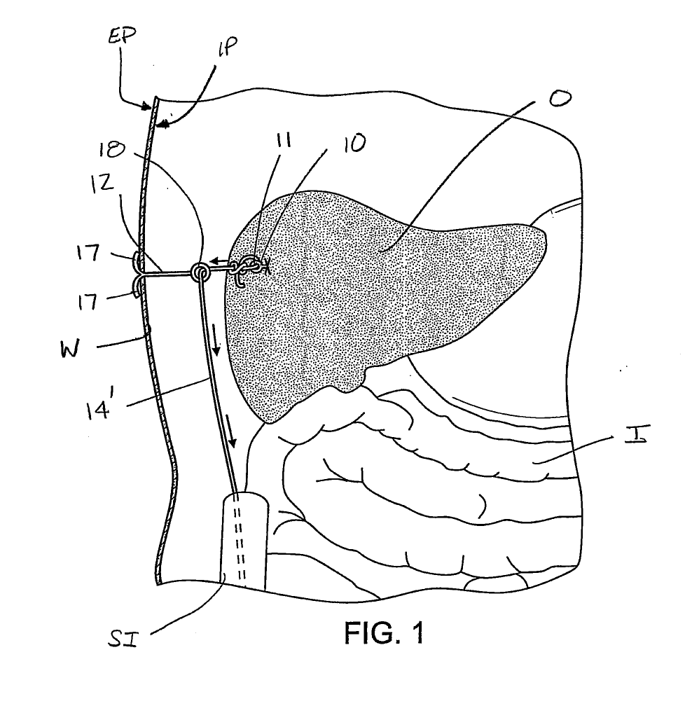

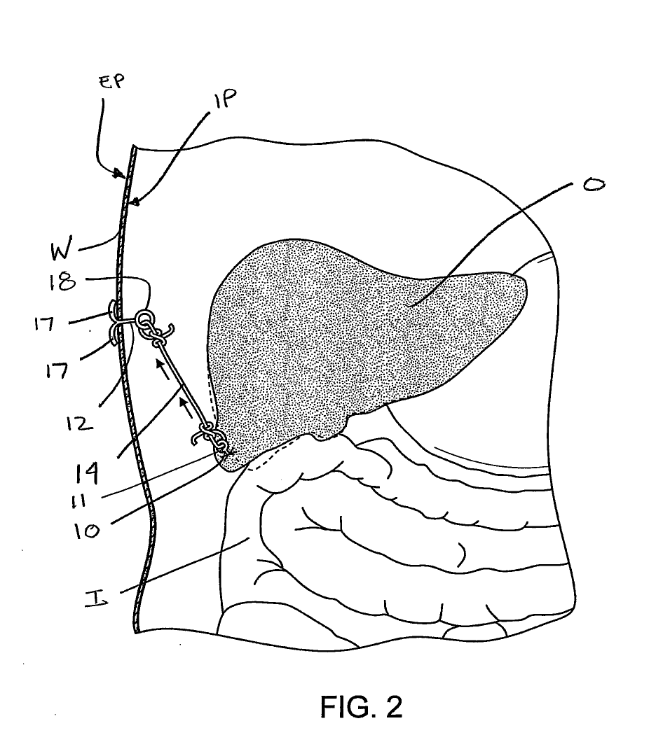

In various embodiments, a surgical device kit can include components which can be configured to manipulate tissue and create a working space within the body of a patient such that a surgeon can easily access, and perform work within, a surgical site using various surgical instruments. In at least one embodiment, referring to FIGS. 1 and 2, a surgical device kit can include several components, such as anchor 10, hanger 12, and connection member 14, for example, which can be cooperatively utilized in order to create the working space within the body. In various embodiments, anchor 10 can be configured to be engaged with tissue at a first location within the patient's body, wherein, in at least one embodiment, the tissue can comprise an organ “O”. In various embodiments, anchor 10 can include a suture, a pin, a band, an adhesive, a clip, and/or any other suitable anchor member configured to retain anchor 10 to the tissue. In at least one embodiment, a first end of anchor 10 can include a retaining member configured be engaged with, or inserted into, the tissue such that the anchor can remain attached to the tissue when a force is applied thereto as described in further detail below. In various embodiments, the second end of anchor 10 can include connector 11 which can be configured to be operably engaged with connection member 14. In at least one embodiment, connector 11 can comprise an eyelet which can be utilized to tie an end of connection member 14 to anchor 10. In certain embodiments, at least one of an anchor, a hanger, a connection member, and/or another suitable component of the surgical kit can be at least partially comprised of a bioabsorbable, or dissolvable, material. Various components can also be at least partially comprised of a non-bioabsorbable, or non-dissolvable, material. In some embodiments, at least one of an anchor, a hanger, and a connection member can be at least partially comprised of an imagable material, i.e., a material which can be viewed by an X-ray device, an ultrasound device, and/or any other suitable imaging device. In various embodiments, the imagable material can be at least partially comprised of barium sulfate, for example.

In various embodiments, hanger 12 of the surgical device kit can be configured to be engaged with tissue at a second location within the patient's body, wherein, in at least one embodiment, the tissue can comprise cavity wall “W”, such as an abdominal wall, for example. In at least one embodiment, hanger 12 can include a base having a first end and a second end where the first end can be configured to pierce and be retained within wall W. In at least one such embodiment, the first end of the base can be positioned on one side of an external portion “EP” of the wall and the second end of the base can be positioned on the other side of an internal portion “IP” of the wall. In various embodiments, the first end of the base can include one or more projections 17 extending perpendicular and/or transverse to the base wherein projections 17 can be configured to engage the external portion of the wall and prevent, or at least inhibit, hanger 12 from being pulled into the body when a force is applied to hanger 12. In other various embodiments, hanger 12 can have any other suitable shape configured to retain hanger 12 to the cavity wall, for example. In at least one embodiment, as described in greater detail below, the second end of the base can include connector 18 which can be operably engaged with connection member 14. In various embodiments, similar to the above, connector 18 can comprise an eyelet.

In various embodiments, referring to FIG. 2, a first end of connection member 14 can be attached to connector 11 of anchor 10, as outlined above. In at least one embodiment, connection member 14 can be comprised of suture thread, for example, such that the surgeon can tie one end of the suture thread to connector 11 of anchor 10. In at least one embodiment, connection member 14 can comprise a resilient, or elastic, material, such as a rubber cord, for example. In any event, the surgeon can thread an end of connection member 14 through the eyelet of connector 18 of hanger 12, and/or wrap a portion of connection member 14 around hanger 12, and apply a force to, or pull, connection member 14 such that anchor 10, and the tissue engaged therewith, is moved toward hanger 12. In various embodiments, connection member 14 can be used to lift the tissue, such as organ O, for example, away from other tissue within and/or proximate to the surgical site, such as intestine “I”, for example, thereby creating working space for the surgeon. In at least one embodiment, the distance between anchor 10 and hanger 12 can be adjusted by shortening or increasing the length of connection member 14 between anchor 10 and hanger 12 such that, in effect, the tissue and/or organ can be retained or biased a desired distance away from the surgical site, for example. Thereafter, connection member 14 can be knotted, or otherwise secured, to hanger 12. In at least one embodiment, a plurality of connection members can be provided to suspend the tissue. In various embodiments, a plurality of connection members can be provided wherein at least some of the connection members can have different moduli of elasticity such that they can be stretched different amounts.

In various embodiments, a surgical device kit can include a surgical instrument, such as surgical instrument “SI”, for example, wherein surgical instrument SI can comprise an endoscope, for example. In at least one embodiment, referring to FIG. 1, a first end of connection member 14′ can be attached to anchor 10 and a portion of connection member 14′ can be threaded through the eyelet of connector 18 wherein, in addition to the above, a second end of connection member 14′ can be operably engaged with the surgical instrument. In various embodiments, the second end of connection member 14′ can extend through a working channel in the surgical instrument such that the second end of connection member 14′ can be pulled relative to hanger 12, for example. In such circumstances, the force applied to connection member 14′ can displace connection member 14′ and shorten the distance between anchor 10 and hanger 12. In various embodiments, the connection member can be mounted to a surgical instrument such that, when the surgical instrument is pulled away from hanger 12, the distance between anchor 10 and hanger 12 can decrease and, when the surgical instrument is moved toward hanger 12, the distance between anchor 10 and hanger 12 can increase. In at least one such embodiment, the tissue engaged by anchor 10 can be moved a distance which is proportional to the distance in which the surgical instrument is moved relative to hanger 12, for example. In various embodiments, although not illustrated, the connector of the hanger can include a rotatable member or pulley which can facilitate the movement of the connection member relative to the hanger.

In various embodiments, the hanger can include an integral and/or attachable member configured to be engaged with at least a portion of a surgical instrument. In at least one embodiment, the member can be used to hold at least a portion of the surgical instrument during a surgical procedure, for example. In various embodiments, the member can include a snap, slot, clip, hook, thread, locking J-slot, band and/or a string, for example. In at least one embodiment, the member can be used to attach and/or suspend an endoscope, a light fiber, a suction device, and/or an instrument hanger, for example, from the member. In certain embodiments, the member can be at least partially comprised of a bioabsorbable, non-bioabsorable, dissolvable, and/or non-dissolvable material, for example.

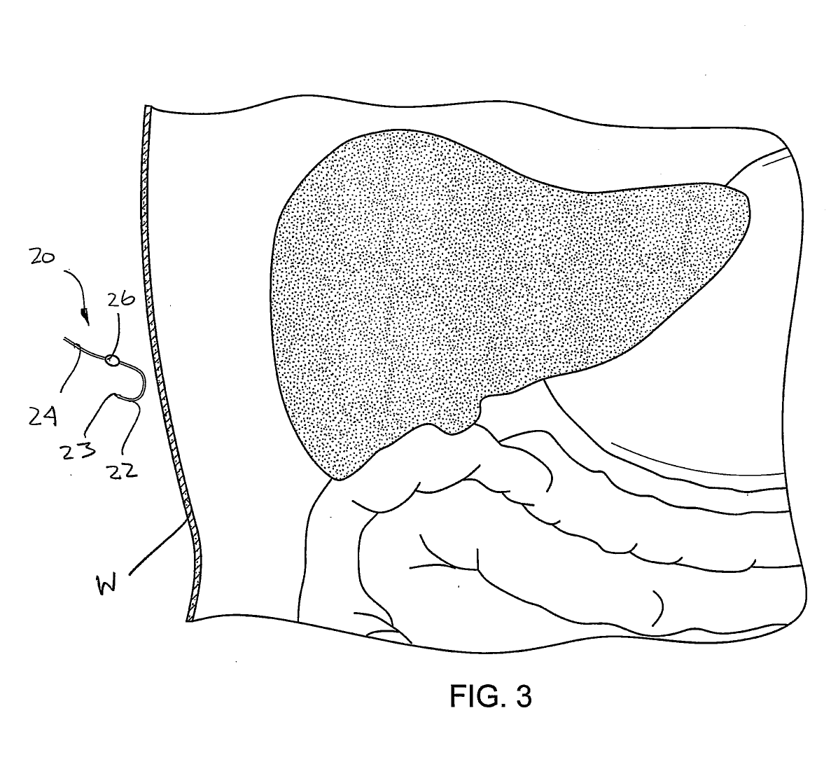

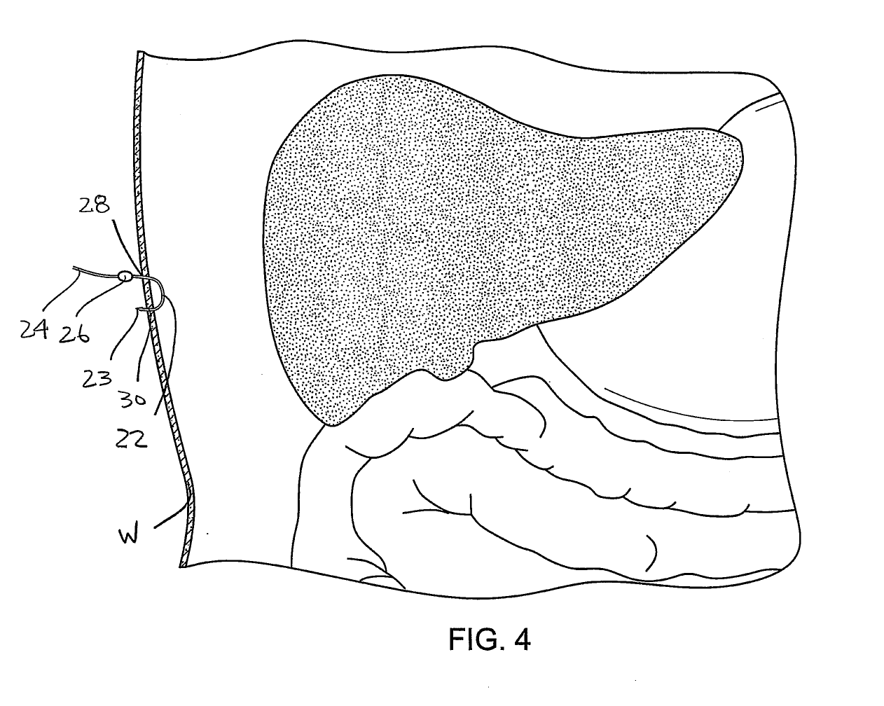

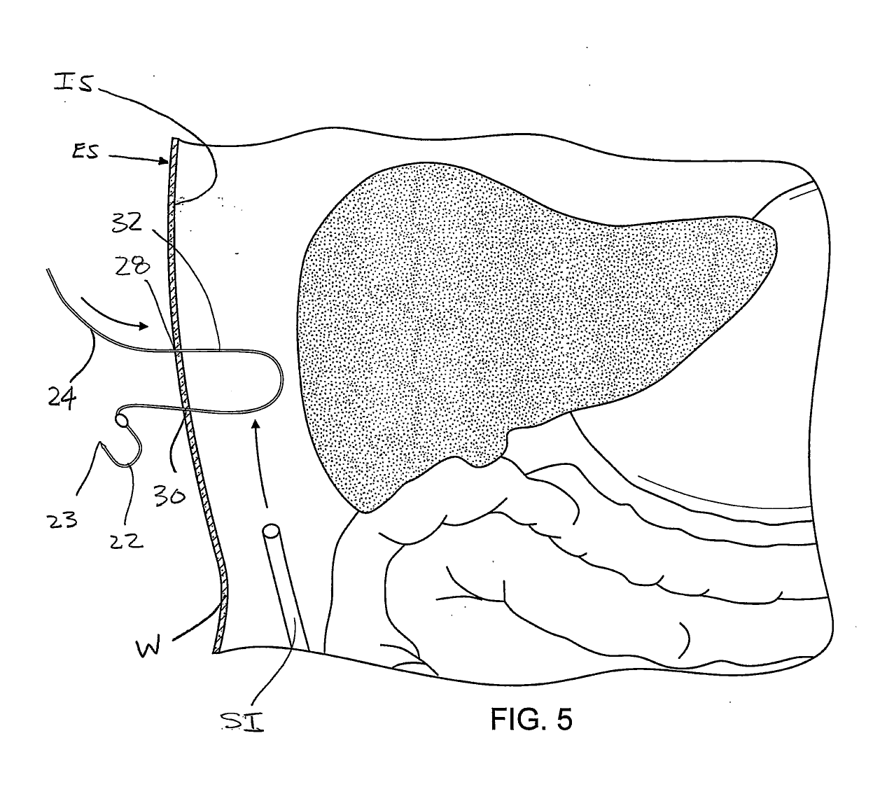

In various embodiments, a surgical device kit can include components which can be configured to support a surgical instrument positioned within the body of a patient. In at least one embodiment, referring to FIGS. 3-7, surgical instrument hanger 20 can include needle 22, or any other suitable tissue-piercing member, and flexible wire 24 having first end 26 attached to needle 22 wherein, in at least one embodiment, wire 24 can be configured to be threaded through an aperture in needle 22. In various embodiments, needle 22 can include tissue-piercing tip 23 which can be configured to pierce a cavity wall “W” of the body. In at least one embodiment, needle 22 can be semi-circular or arcuate in shape, for example. In use, a surgeon can use needle 22 to insert wire 24 through a portion of wall W in a suturing-like motion, for example. In such circumstances, referring to FIG. 4, the surgeon can insert needle 22 through wall W at first point 28 into the abdominal or peritoneal cavity, for example, and, as also illustrated in FIG. 4, the surgeon can reinsert needle 22 into wall W at second point 30 to remove needle 22 from the cavity as illustrated in FIG. 5. In various embodiments, a needle may not be needed to insert wire 24 through a cavity wall. In at least one such embodiment, wire 24 can be comprised of a stiff suturing thread, a malleable material, and/or any other suitable semi-rigid material, which can be suitably bent and inserted through wall W, for example. In any event, as a result of the above, at least a portion of wire 24 can remain in the cavity and, in at least one embodiment, form loop 32 therein.

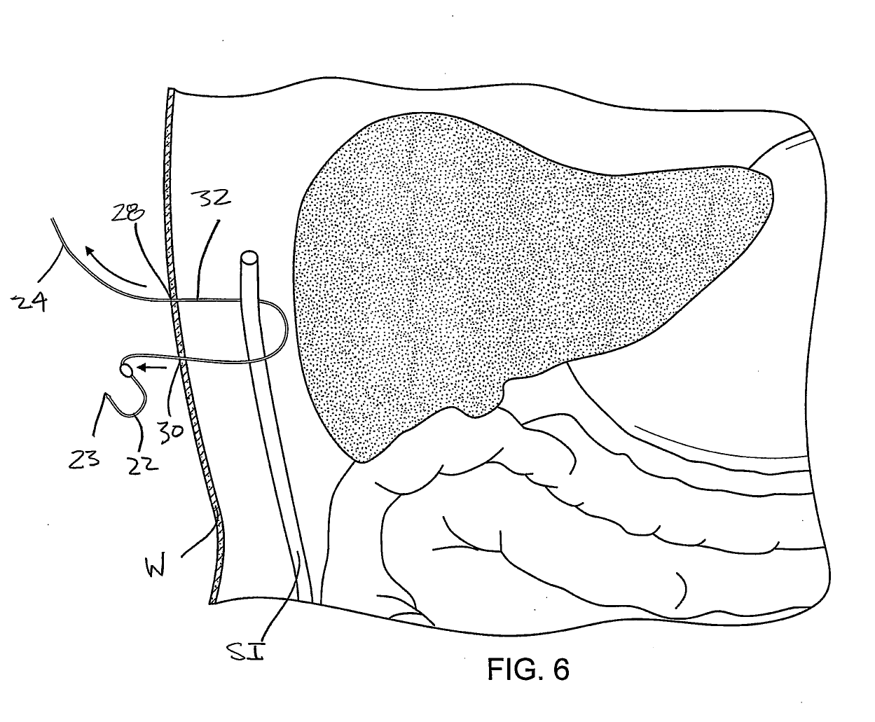

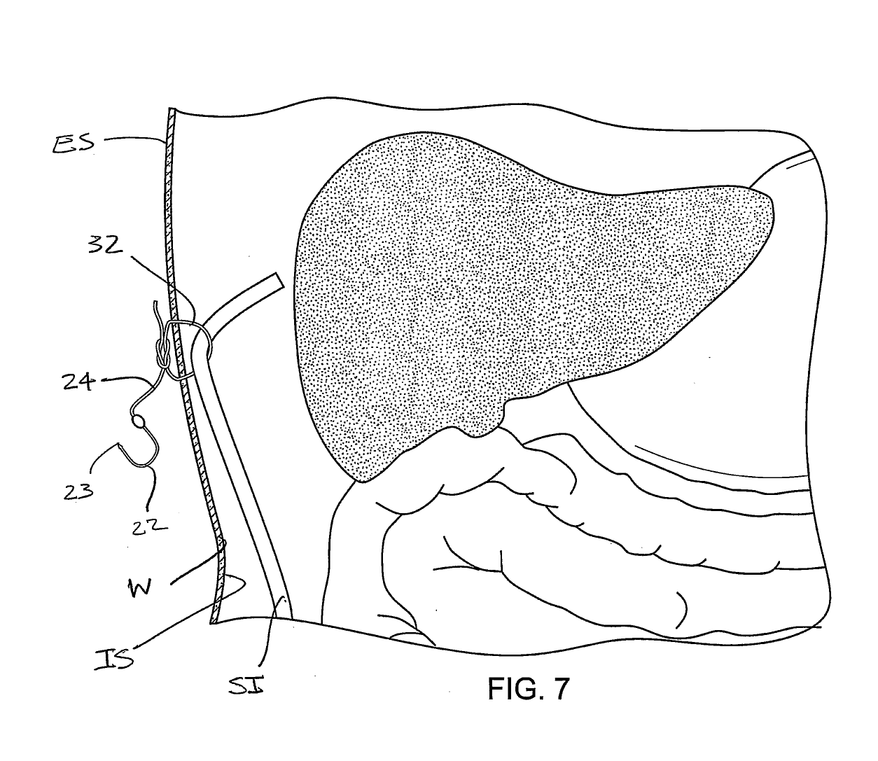

In various embodiments, referring to FIGS. 5 and 6, loop 32 can be large enough to at least partially surround and/or engage a surgical instrument “SI” positioned within the body. In at least one embodiment, the surgical instrument can include an overtube and/or endoscope, for example, which can be inserted through loop 32. In at least one embodiment, referring to FIG. 7, a surgeon can apply a force to at least one of the ends of wire 24 such that the size of loop 32 can be reduced and, in various embodiments, the surgical instrument can be held against, or in close proximity to, the inner surface, IS, of the cavity wall. In various embodiments, the internal surface of the wall can provide support to the surgical instrument, for example. In various circumstances, the ends of wire 24 can be manipulated to hold the surgical instrument in position wherein, in at least one embodiment, the ends of wire 24 can be twisted, tied, or otherwise engaged with one another. In various embodiments, at least a portion of the wire can be bent and/or folded over to contact an external surface “ES” of the wall which can then be taped, glued, and/or otherwise secured to the external surface to hold the surgical instrument in a suitable position.

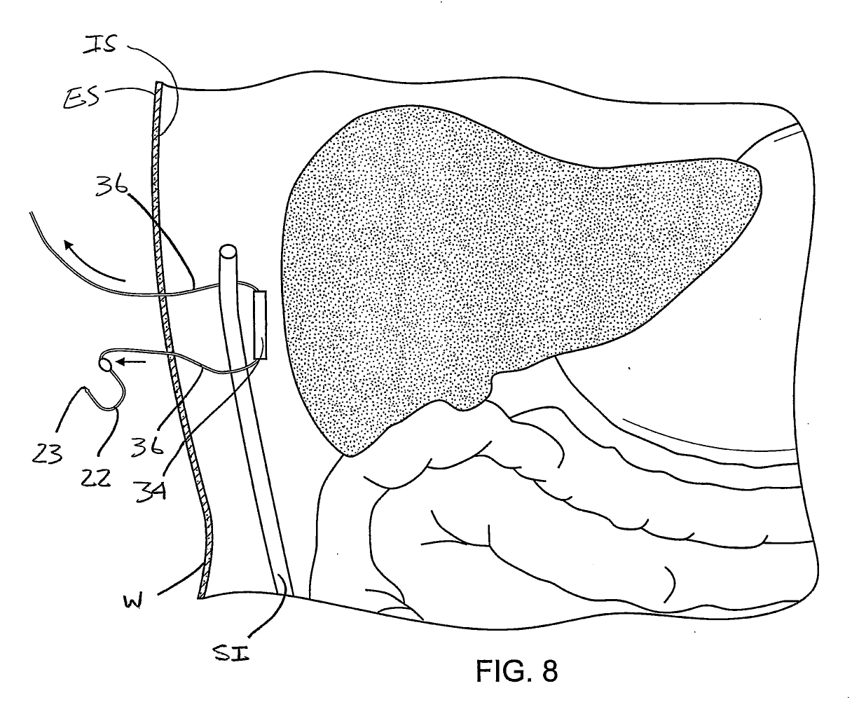

In various embodiments, similar to the above, a surgical device kit can include components which can be configured to support a surgical instrument positioned within the body of a patient. In at least one embodiment, referring to FIG. 8, the surgical device kit can include support member 34 which can be inserted into a cavity via a natural opening in the patient's body, a trocar, an endoscope, and/or through a small incision. In various embodiments, support member 34 can include a tissue-piercing tip which can be configured to pierce the wall “W” of the body to allow support member 34 to be inserted directly into the cavity, for example. In at least one embodiment, support member 34 can be suspended or hung from the wall of the cavity by at least one hanger 36, for example. In various embodiments, hangers 36 can be comprised of suture thread, wire, and/or any other suitable material, for example, wherein hangers 36 can be mounted to, or otherwise engaged with, support member 34. In at least one embodiment, a surgeon can attach a hanger 36 to the cavity wall by utilizing an anchor mounted to the wall, for example. In other various embodiments, hanger 36 can be configured to extend through the wall and can be folded and/or bent such that it can be secured to the external surface “ES” of the wall using an adhesive and/or surgical tape, for example. In various embodiments, similar to the above, a surgical instrument, such as an overtube and/or an endoscope, for example, can be retained to, or suspended from, the support member 34. In various embodiments, member 34 can include a plurality of hooks, hangers, clips, and/or apertures configured to engage at least a portion of a surgical instrument “SI” positioned within the body to aid in retaining the surgical instrument to support member 34. In various embodiments, support member 34 can be comprised of a rigid or a semi-rigid material and can be removed from the body after a surgical procedure. In other various embodiments, support member 34 can be comprised of a dissolvable, bioabsorbable, biofragmentable, and/or therapeutic material such that the member can remain within the body until it is absorbed and/or dissolved by the body.

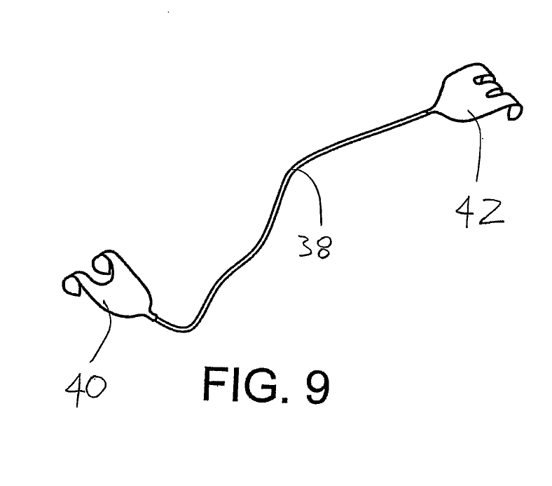

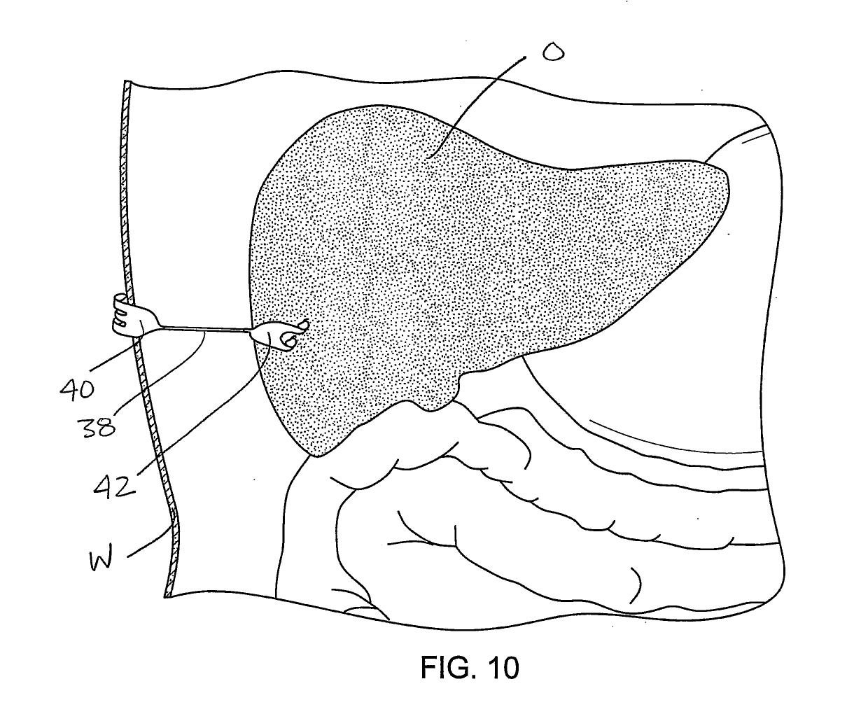

In various embodiments, referring to FIGS. 9 and 10, a surgical device kit can include components which can be configured to hold tissue, such as organ O, for example, relative to the wall of a body cavity in order to create working space within a surgical site. In at least one embodiment, referring to FIG. 9, the surgical device kit can include a hanger comprising connection member 38, first attachment member 40, and second attachment member 42. In various embodiments, connection member 38 can include a first end and a second end, wherein first attachment member 40 can be mounted to the first end and second attachment member 42 can be mounted to the second end. In at least one embodiment, the first and second attachment members can include clips, pins, clamps, bands, anchors, sutures, and/or any other suitable attachment member configured to be engaged with the tissue, an organ, and/or sidewall of a cavity. More particularly, referring to FIG. 10, first attachment member 40 can be configured to be inserted through an incision in sidewall W, for example, and second attachment member 42 can be configured to engage organ O, such as a liver or gall bladder, for example, positioned proximate to the surgical site. In various embodiments, connection member 38 can be comprised of suture thread, an elastic cord, and/or any other suitable material.

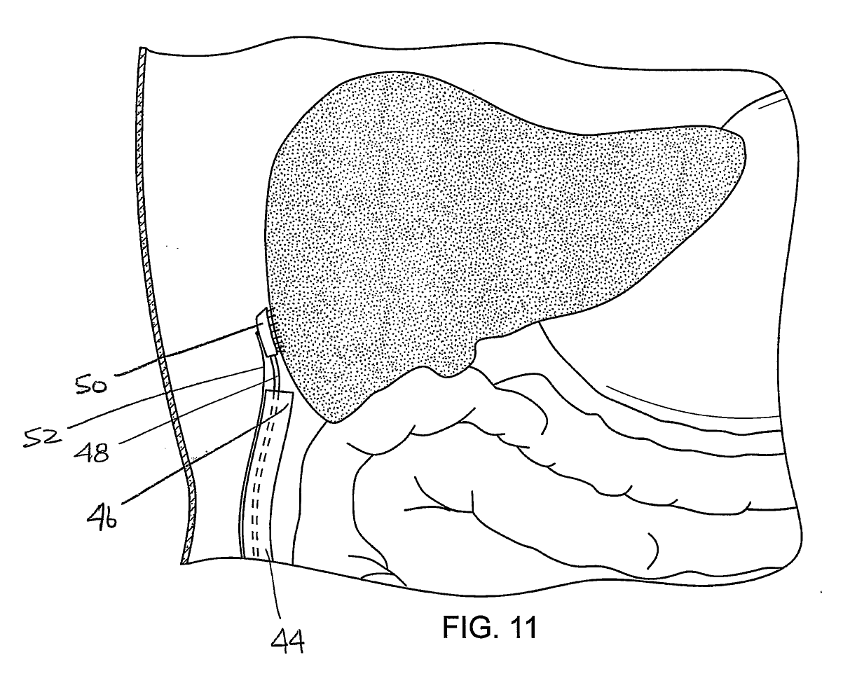

In various embodiments, similar to the above, a surgical instrument can be used to manipulate tissue within the body of a patient to create a working space for a surgeon within a surgical site. In at least one embodiment, referring to FIG. 11, a surgical instrument can include a first member, such as overtube 44, for example, having a proximal end (not illustrated) and distal end 46. In such an embodiment, the proximal end can be configured to be positioned external to the patient's body while the distal end can be configured to be positioned within the surgical site. In various embodiments, referring to FIG. 11, the surgical instrument can further include a second, or tissue-contacting, member which can be moved relative to overtube 44. In at least one embodiment, tissue-contacting member 50 can be operably engaged with overtube 44 such that member 50 can be slid, or translated, relative to overtube 44 between first and second positions. In order to move member 50 between its first and second positions, the surgical instrument can further include extension tube 48 operably engaged with tissue-contacting member 50 such that, when extension tube 48 is motivated by a surgeon, for example, member 50 can be motivated by extension tube 48. In various embodiments, extension tube 48 can include an aperture, or channel, therein which can be placed in fluid communication with a vacuum source and at least one aperture in tissue-contacting member 50. In at least one such embodiment, tissue-contacting member 50 can be positioned on, and in sealed fluid communication with, a distal end of extension tube 48. In any event, in at least one embodiment, a vacuum having a negative fluid pressure can be supplied to the channel in extension tube 48 and communicated to the aperture in tissue-contacting member 50 such that tissue can be held to member 50. In various circumstances, member 50 can be configured to move the tissue when member 50 is moved between its first and second positions.

In various embodiments, tissue-contacting member 50 can be articulated relative to distal end 46 of overtube 44, for example, in order to manipulate the tissue and create a working space within the surgical site. In at least one embodiment, the surgical instrument can further include articulation member 52 which can be operably engaged with tissue-contacting member 50. In at least one embodiment, articulation member 52 can include a wire and/or any other suitable flexible and/or semi-rigid member, for example. In various embodiments, a force can be applied to articulation member 52 in order to pull articulation member 52 distally and, in at least the illustrated embodiment, rotate tissue-contacting member 50 upwardly. In at least one such embodiment, articulation member 52 can extend along at least a portion of overtube 44 such that a surgeon can motivate articulation member 52 from a location outside of the patient's body, for example. Although only one articulation member is illustrated in the exemplary embodiment, more than one articulation member can be utilized to articulate tissue-contacting member 50 in a plurality of directions with respect to overtube 44, for example.

In various embodiments, referring to FIG. 13, a surgical instrument can include an overtube which can be secured to an endoscope, for example. In at least one embodiment, overtube 44 can be snap-fit, press-fit, and/or otherwise suitably mounted to endoscope 53 such that overtube 44 does not move, or at least substantially move, relative to endoscope 53. In various embodiments, overtube 44 can include cap 61 and tissue-contacting member 50, wherein cap 61 can be mounted to endoscope 53 and tissue-contacting member 50 can be rotatably mounted to cap 61. In at least one embodiment, similar to the above, the surgical instrument can further include extension tube 65 having a channel therein which can be placed in fluid communication with a vacuum source and at least one aperture in tissue-contacting member 50. In various embodiments, tissue-contacting member 50 can be rotated or pivoted about pin 63 between at least first and second positions by articulation member 52 wherein, when a vacuum is supplied to the apertures in member 50, member 50 can manipulate tissue within and/or adjacent to the surgical site.

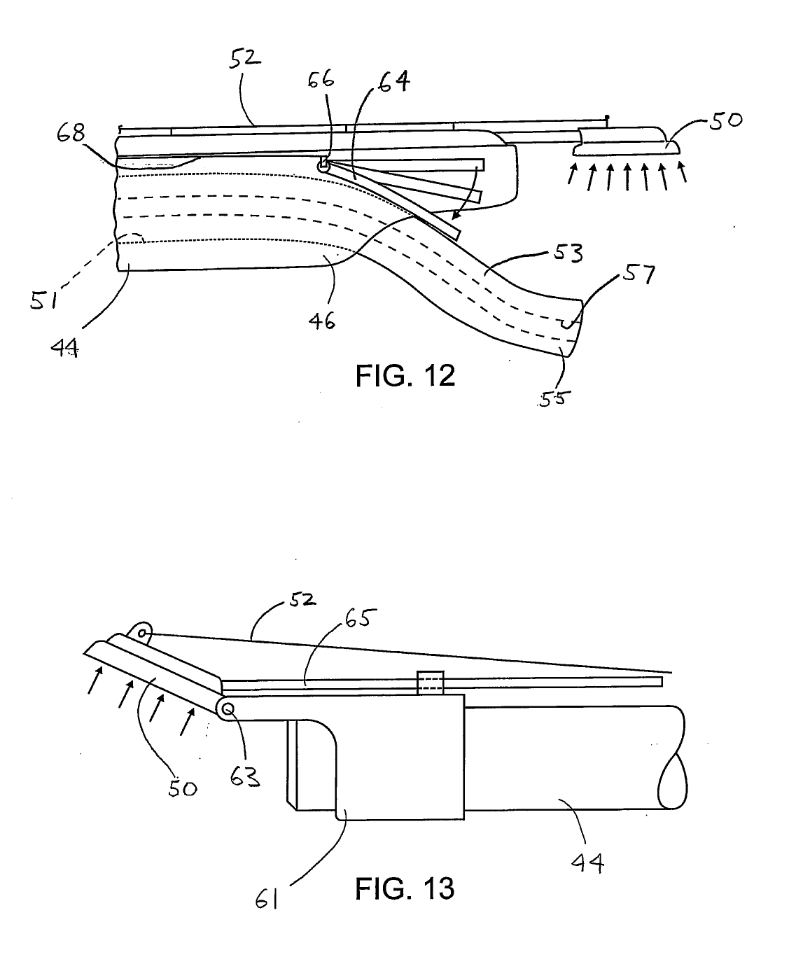

In various embodiments, a surgical instrument can further include an aperture extending therethrough which can be configured to receive another surgical instrument therein. In at least one embodiment, referring to FIG. 12, a surgical instrument, such as overtube 44, can include aperture 51 extending therethrough which can be configured to receive endoscope 53. In various circumstances, endoscope 53 can be slid within aperture 51 between at least a first position and a second position in order to locate and/or orient endoscope 53 within the surgical site. In various embodiments, distal end 46 of overtube 44 can include instrument director 64 which can be configured to direct endoscope 53, and/or another surgical instrument, in various directions when endoscope 53 extends through the distal end of the overtube. In at least one embodiment, the instrument director can be configured to contact and push the surgical instrument against the side of aperture 51, for example. In various embodiments, instrument director 64 can be configured to orient endoscope 53, for example, in a downward direction, or any other suitable direction, such that, when endoscope 53 is articulated, endoscope 53 can be articulated such that end 55 of endoscope is directed, or at least substantially directed, toward tissue-contacting member 50. In such embodiments, owing to the configuration of endoscope 53, a camera within end 55 can be utilized to observe the tissue being manipulated by tissue-contacting member 50.

In various embodiments, referring again to FIG. 12, a surgical instrument can include at least one instrument director, such as instrument director 64, for example, which can include a first end rotatably attached to an overtube and a second end configured to contact an instrument, such as an endoscope, for example, positioned within overtube 44. In at least one embodiment, the surgical instrument can further include at least one spring, or spring element, configured to bias instrument director 64 into engagement with endoscope 53. In such circumstances, further to the above, the instrument director can prevent, or at least inhibit, fluid and/or tissue from entering into the overtube. In at least one such embodiment, after endoscope 53 has been retracted into aperture 51, the spring can bias instrument director 64 into a position such that the end of aperture 51 can be closed, or at least substantially closed, such that fluid and/or tissue can be prevented, or at least inhibited, from entering into aperture 51. In at least one embodiment, the first end of instrument director 64 can include projection 66 extending therefrom which can be operably engaged with actuator 68. In various embodiments, actuator 68 can extend along the length of the overtube such that a surgeon can pull and/or push actuator 68 to rotate instrument director 64 relative to distal end 46 of overtube 44.

Further to the above, in various embodiments, an endoscope, such as endoscope 53, for example, can include a working channel, such as working channel 57, for example, which can be configured to receive a second surgical instrument therein. In various embodiments, the second surgical instrument can include graspers, a cutting instrument, a harmonic instrument, and/or a stapling instrument, for example, which can be configured to treat tissue within and/or surrounding a surgical site. In at least one embodiment, referring to FIG. 13, the second surgical instrument (not illustrated) and tissue-contacting member 50 can be cooperatively utilized to manipulate the tissue in order to create working space within the surgical site. In various embodiments, tissue-contacting portion 50, for example, can be used to engage, push, pull, lift, and/or bias tissue relative to the distal end 46 of overtube 44 while the second surgical instrument can simultaneously cut, or otherwise treat, the tissue. In various embodiments, although not illustrated, the distal end of overtube 44 can include a seal which can be configured to sealingly engage the endoscope and/or the shaft of the second surgical instrument when the scope and/or second surgical instrument exits the distal end of the overtube to access the surgical site.

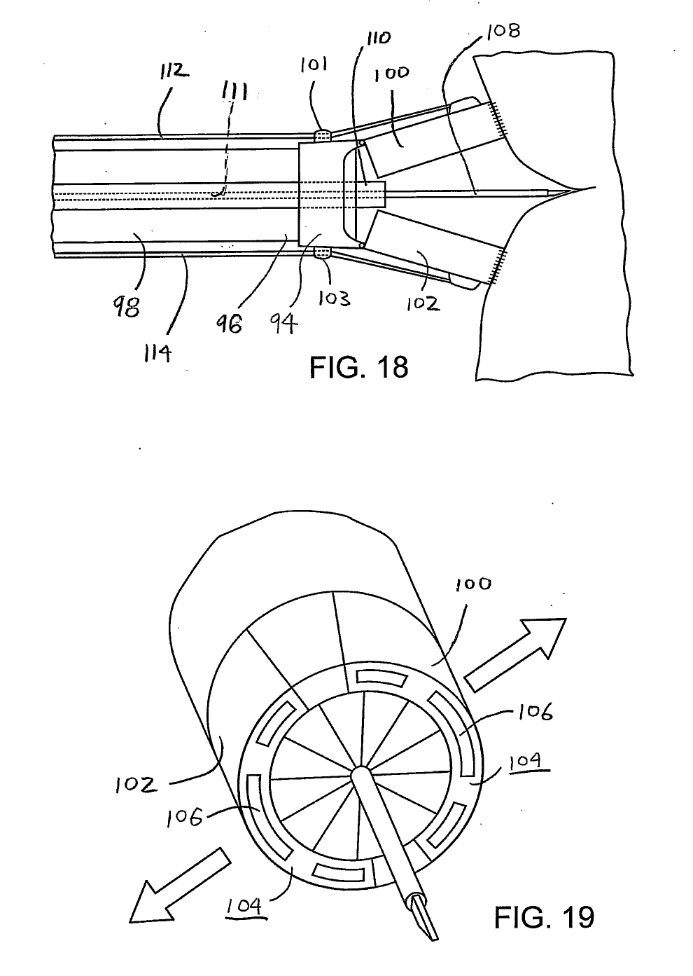

In various embodiments, further the above, a surgical instrument in accordance with at least one embodiment of the present invention can include two or more tissue-contacting members. In at least one embodiment, referring to FIGS. 18 and 19, a surgical instrument can include manifold 94 which can be positioned on distal end 96 of overtube 98, where manifold 94 can be configured to supply tissue-contacting members 100 and 102 with a vacuum, or negative fluid pressure. More particularly, in at least one embodiment, overtube 98 can include an aperture or channel therein which can be in fluid communication with manifold 94 where, in at least one embodiment, manifold 94 can further include channels, apertures, and/or tubes which can communicate the vacuum to first tissue-engaging member 100 and second tissue-engaging member 102. In various embodiments, similar to the above, tissue-engaging members 100 and 102 can each include at least one aperture therein which can allow the vacuum to be communicated to tissue within the surgical site such that the tissue can be retained thereto. In various embodiments, referring to FIG. 19, the distal ends of first and second members 100 and 102 can each include a face 104 and a plurality of openings, or apertures, 106 defined therein, where openings 106 can be in fluid communication with manifold 94 as described above. In use, referring to FIG. 18, faces 104 of tissue-contacting members 100 and 102 can be positioned against tissue within and/or adjacent to a surgical site such that portions of the tissue are held thereto by the vacuum. Thereafter, first and second members 100 and 102 can be moved relative to each other to move, and/or separate, the portions of tissue.

In at least one embodiment, the first and second tissue-engaging members can include proximal ends which can be rotatably mounted to manifold 94 such that the first and second tissue-engaging members 100 and 102 can be pivoted with respect to each other and/or overtube 98. In various embodiments, the first and second tissue-engaging members 100 and 102 can be configured to separate layers of tissue, and/or tissue from an organ, when members 100 and 102 are moved apart from each other. More particularly, in at least one embodiment, the first and second tissue-engaging members 100 and 102 can engage separate layers of tissue while they are positioned proximate to each other and can then be pivoted away from each other to thereby create a working space between the layers of tissue. In various embodiments, the surgical instrument can further include first articulation member 112 for rotating first member 100 about pin 101 and, in addition, second articulation member 114 for rotating second member 102 about pin 103. In various embodiments, referring to FIGS. 18 and 19, a second surgical instrument, which can include a cutting member, for example, can be inserted through an aperture in the surgical instrument in order to cut and/or separate the layers of tissue. In various embodiments, second surgical instrument 108 can be positioned intermediate first and second tissue-engaging portions 100 and 102 such that second surgical instrument 108 can dissect the layers of tissue, for example. In at least one embodiment, referring to FIG. 18, second surgical instrument 108 can be positioned within working channel 111 of endoscope 110 such that it can be extended and/or retracted relative to endoscope 110.

In various embodiments, further to the above, the first and second articulation members 112 and 114 can include wires, and/or other rigid or semi-rigid members, which can each extend from their respective tissue-engaging members along the length of overtube 98 such that articulation members 112 and 114 can be accessible to a surgeon. In at least one embodiment, guides can be provided along the length of the tissue-engaging portions and/or overtube in order to control the movement of the articulation members. In various embodiments, the articulation members can be operably engaged with one or more handles, buttons, or levers (not illustrated) positioned on, or near, the proximal end of the overtube such that the articulation members can be actuated by a surgeon. In various embodiments, a surgeon can selectively actuate the articulation members such that at least one of the tissue-contacting members is moved while at least one of the articulation members remains stationary. In at least one embodiment, articulation members 112 and 114 can be comprised of wires which can be sufficiently rigid such the wires can be pushed towards distal end 96 of overtube 98 to apply a closing force, for example, to the first and second tissue-engaging members. Articulation members 112 and 114 can also be pulled proximally in order to apply an opening force to the first and second tissue-engaging members 100 and 102. In other various embodiments, the first and second tissue-engaging portions can be actuated pneumatically, hydraulically, mechanically, and/or electrically using any suitable devices.

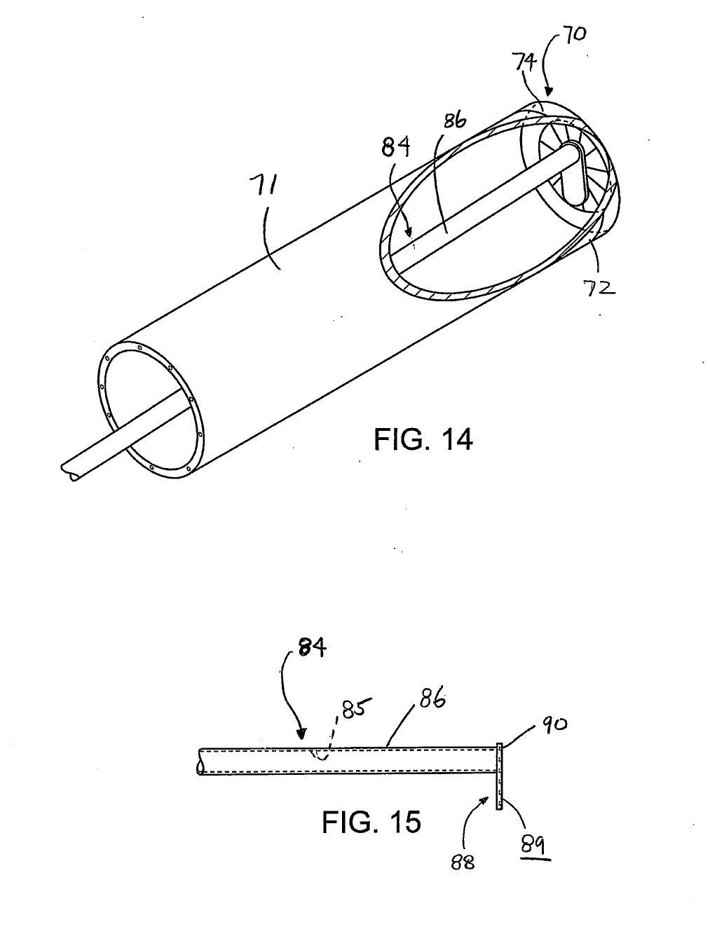

In various embodiments, a surgical instrument kit can include a first tissue-contacting member configured to hold tissue in position and, in addition, a second tissue-contacting member which can be moved relative to the first tissue-contacting member. In at least one embodiment, a surgical instrument can include, referring to FIG. 14, a first member, such as overtube 71, for example, and a second member, such as rod 84. In various embodiments, overtube 71 can include manifold 70 attached to, or integrally formed with, distal end 72. In at least one embodiment, referring to FIGS. 14 and 17, manifold 70 can include body portion 74 which can be configured to engage distal end 72 of overtube 71. In addition, manifold 70 can further include tissue-contacting surface 76 which can be configured to engage tissue positioned within and/or proximate to a surgical site. In various embodiments, overtube 71 can further include at least one aperture or channel 73 which can be placed in fluid communication with a vacuum or a negative fluid pressure source. In at least one embodiment, manifold 70 can be in fluid communication with channel 73 in overtube 71 such that the vacuum can be communicated to at least one opening 78 defined within tissue-contacting surface 76, for example. In at least one such embodiment, the vacuum communicated to openings 78 can be sufficient to hold tissue to tissue-contacting surface 76 and, once manifold 70 is engaged with the tissue, the surgeon can manipulate overtube 71 to thereby manipulate the tissue. In various alternative embodiments, a positive fluid pressure supply can be placed in communication with channel 73 in overtube 71 such that tissue can be pushed away from manifold 70.

In various embodiments, manifold 70 can further include port 80 defined therein, wherein port 80 can be configured to receive a second member, or rod 84, therethrough. In at least one embodiment, referring to FIGS. 14-16, rod 84 can include a first end, a second end, and shaft 86 extending intermediate the first and second ends where the shaft can include a channel or aperture 85 extending therethrough. In at least one embodiment, similar to the above, channel 85 in rod 84 can be placed in fluid communication with one of a negative and a positive fluid pressure source or pump (not illustrated). In various embodiments, rod 84 can further include end-effector 88 which can include at least one tissue-contacting surface 89 and at least one aperture 90 therein. Similar to the above, apertures 90 can be in fluid communication with channel 85 in shaft 86 such that tissue can be retained to end-effector 88 by a vacuum supplied thereto. In various embodiments, shaft 86 can define an axis and end-effector 88 can extend in a direction which is transverse, perpendicular, and/or oblique to the axis of shaft 86, for example. In any event, the second member, or rod 84, can be utilized to grip tissue within and/or adjacent to a surgical site such that a surgeon can manipulate rod 84 in order to manipulate the tissue. In various embodiments, a vacuum supplied to overtube 71 can be utilized to hold tissue at a first location, a vacuum supplied to rod 84 can be utilized to hold tissue at a second location, wherein rod 84 can be moved relative to overtube 71 to separate or make working space between the tissue.

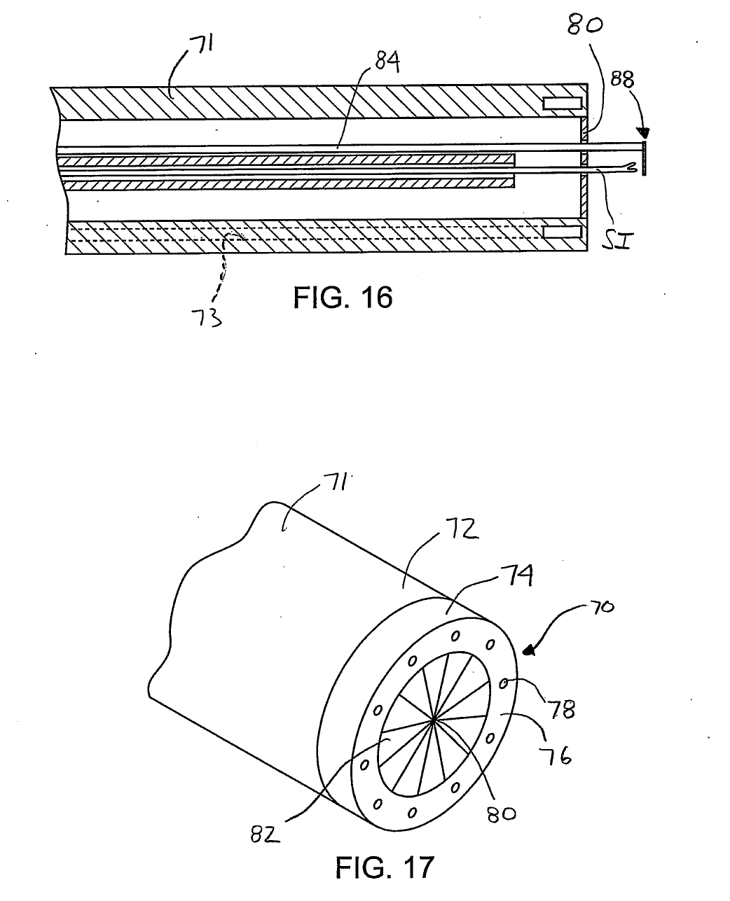

In various embodiments, overtube 71 can further be configured to receive a surgical instrument therethrough and allow the surgical instrument to access the surgical site. In at least one embodiment, referring to FIGS. 16 and 17, overtube 71 can further include seal 82 which can be configured to sealingly engage at least a portion of the surgical instrument, such as surgical instrument SI, for example, when the surgical instrument is extended through port 80. Such seals can include the seals disclosed in U.S. Pat. No. 5,401,248, entitled SEAL FOR TROCAR ASSEMBLY, which issued on Mar. 28, 1995, U.S. Pat. No. 5,628,732, entitled TROCAR WITH IMPROVED UNIVERSAL SEAL, which issued on May 13, 1997, and U.S. Pat. No. 5,792,113, entitled UNIVERSAL SEAL FOR A TROCAR, which issued on Aug. 11, 1998, the entire disclosures of which are hereby incorporated by reference. Once the surgical instrument has been positioned within the surgical site, the surgical instrument can be used in cooperation with the first and second members outlined above in order to manipulate and treat the tissue.

In lieu of overtube 71 described above, in various embodiments, an overtube can be mounted to an endoscope. In at least one embodiment, the overtube can be snap-fit, press-fit, and/or otherwise suitably mounted to the endoscope. In various embodiments, the endoscope can include an aperture or channel therein which can be placed in fluid communication with a vacuum source, for example, and the overtube can comprise a manifold which, when the overtube is mounted to the endoscope, can be in fluid communication with the channel. In at least one embodiment, the manifold can define a perimeter of apertures which can be configured to communicate the vacuum to the tissue in the surgical site. In various embodiments, the endoscope can include at least one working channel and the manifold can include at least one port therein which can allow surgical instruments to be passed through the endoscope and the manifold to allow them to enter the surgical site.

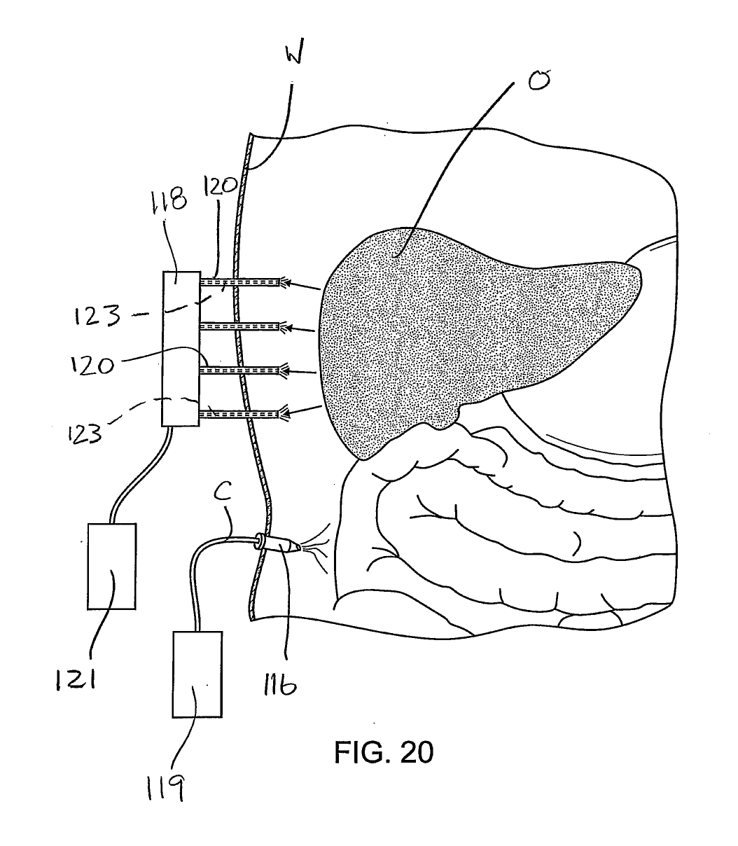

In various circumstances, as outlined above, a cavity within the patient's body, such as the abdominal or peritoneal cavity, for example, may be insufflated so as to move the wall of the cavity away from the organs therein. In various embodiments, referring to FIG. 20, a trocar, such as cannula 116, for example, can be configured to create, and/or be inserted through, an aperture or incision in wall “W” of the body cavity wherein a conduit “C”, and/or any other suitable pressurization instrument, can be sealingly inserted therein. In at least one such embodiment, the conduit and/or pressurization instrument can be connected to a positive pressure pump positioned external to the body, such as pump 119, for example, wherein the pump can create a positive pressure in the body cavity using a fluid, such as carbon dioxide gas, for example. In at least one embodiment, as outlined above, such a positive pressurization can provide a working space between the wall of the body cavity and the tissue and/or organs within the body cavity.

Further to the above, in various embodiments, a surgical instrument can be used to manipulate tissue within an insufflated body cavity, for example. In at least one embodiment, referring to FIG. 20, a surgical instrument can include base, or manifold, 118, and a plurality of members 120 extending from manifold 118. In various embodiments, members 120 can each have a first end, a second end, and a channel 123 extending between the first and second ends where each second end of members 120 can include at least one opening therein in fluid communication with the channels. In at least one embodiment, manifold 118 can be configured to communicate a negative fluid pressure to channels 123 such that the negative fluid pressure can be communicated through the openings in the second ends and applied to the tissue. In at least one embodiment, members 120 can be parallel, or at least substantially parallel, to one another. In various embodiments, each second end of members 120 can further include a tip configured to pierce the body cavity wall such that a surgeon can position the instrument relative to the patient's body and apply a force to the instrument to insert members 120 into the body cavity as described in greater detail below.

In use, the surgical instrument described above, for example, can be initially positioned external to an insufflated body cavity such that the openings and/or tips of members 120 can be situated adjacent to the wall of the body cavity. In at least one embodiment, the surgeon can make a series of incisions in the body cavity wall such that members 120 can be inserted into the body cavity. In other various embodiments, the surgeon can push manifold 118 towards the wall to cause members 120 to engage and pierce the wall to create apertures therein. In either event, members 120 can sealingly engage the apertures in the body cavity wall to allow the body cavity to remain insufflated with the positive pressure described above. In various embodiments, the surgeon can activate second pump 121, which can be located either internal to or external to manifold 118, for example, to cause pump 121 to communicate a negative fluid pressure, or suction, through the channels and openings in members 120. In at least one embodiment, the suction can be applied to an area proximate to an organ “O” or, alternatively, the suction can be applied directly to organ O to thereby lift and/or otherwise manipulate organ O within the body cavity. In at least one such embodiment, a pressure differential can be created on the opposites sides of an organ, such as the liver, for example, such that the pressure differential can motivate the organ and move it away from a surgical site.

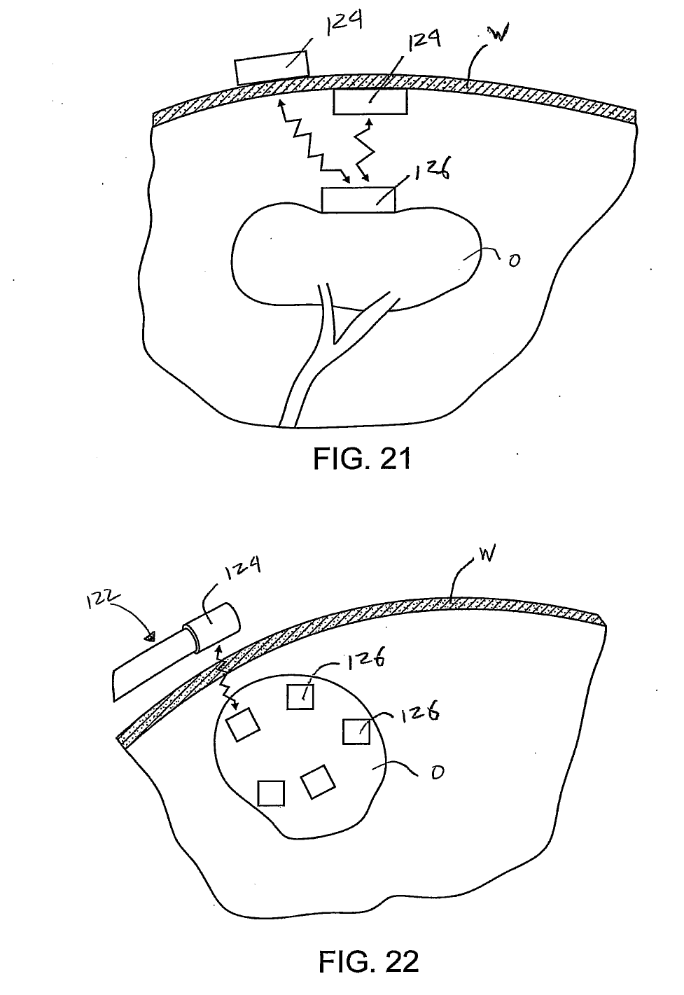

In various embodiments, in addition to or in lieu of the surgical devices described above, magnets and/or magnetic materials can be used to manipulate tissue within a patient's body to create a working space which can allow a surgeon to easily access a surgical site. Various embodiments of such devices are disclosed in commonly-owned, co-pending U.S. patent application Ser. No. 11/622,540, which was filed on Jan. 12, 2007 and is entitled MAGNETIC TISSUE GRASPING, the entire disclosure of which is hereby incorporated by reference herein. In at least one embodiment, a magnetic implant can be engaged with tissue in the patient's body such that a magnet can be utilized to motivate the magnetic implant and manipulate the tissue engaged therewith. In various embodiments, referring to FIG. 21, at least one magnetic implant 126 can be positioned on and/or attached to tissue, such as organ “O”, for example, within the patient's body. Various magnetic implants can be at least partially comprised a rigid magnetic material, a flexible magnetic material, and/or magnetic materials in the form of a gel, such as a magnetic gel manufactured by GELTEC Co., Ltd., for example. In at least one embodiment, magnetic implant 126 can include a magnetic material, such as iron, for example. In addition to or in lieu of the above, magnetic implant 126 can include at least one permanent magnet and/or electromagnet, for example. In various embodiments, at least one magnet 124 can be utilized to attract, or otherwise motivate, magnetic implant 126 relative thereto. More particularly, magnet 124 can be configured to emit at least one magnetic field or magnetic moment which can interact with the magnetic material of magnetic implant 126 and generate a force which can displace magnetic implant 126 and the tissue associated therewith. In various embodiments, the magnetic field emitted by magnet 124, for example, can be configured to either attract and/or repel magnetic implants 126. In at least one embodiment, magnet 124, for example, can be mounted to tissue within and/or adjacent to a surgical site, such as body cavity wall W, for example.

In at least one embodiment, magnet 124 can be moved relative to the patient's body in order to move magnetic implants 126. In various embodiments, referring to FIG. 22, surgical instrument 122 can include a shaft having a handle (not illustrated) positioned on a first, or proximal, end of surgical instrument 122 and, in addition, at least one magnet 124 positioned on a second, or distal, end of surgical instrument 122. In at least one embodiment, magnet 124 can be positioned internal and/or external to wall W of a body cavity, such as the abdominal or peritoneal cavity, for example, to motivate magnetic implants 126 as outlined above. In various embodiments, tissue within a patient's body can be manipulated without magnet 124 and/or surgical instrument 122 contacting the tissue.

In various embodiments, a magnetic implant can be engaged with tissue in at least one of a plurality of ways. In at least one embodiment, magnetic implant 126 can be engaged with tissue through the use of a suture, a clip, an anchor, an adhesive, and/or medical tape, for example. In various embodiments, the magnetic implant can include a tissue-piercing portion, and/or cutting member, configured to create an aperture in the tissue such that the magnetic implant can be slid through the aperture and positioned within the tissue. In various embodiments, a plurality of magnetic implants 126 can be engaged with the tissue such that the tissue can be maneuvered, manipulated, and/or rotated in a variety of directions when a magnetic field interacts with the magnetic implants. In at least one embodiment, the plurality of magnetic implants 126 can be positioned around the perimeter of an organ, for example, such that the organ can easily be manipulated in any suitable direction when a magnet is positioned and moved relative to the organ. In at least one embodiment, a first magnetic implant can be positioned on a first side of the organ and a second magnetic implant can be positioned on a second side of the organ. In various embodiments, the magnets can include positive and negative poles which can be arranged such that, when a certain magnetic field or moment is applied to the magnets, the arrangement of the poles can determine the manner in which the tissue associated with the magnets will move.

In various embodiments, magnet 124 can include an electromagnet and/or a permanent magnet. In at least one embodiment, an electromagnet can include a coil of wire, such as insulated copper wire, for example, wrapped around an iron core, where the iron core can be magnetized when electric current flows through the wire. In at least one embodiment, the amount of electric current flowing through the wire can be proportional to the intensity of the magnetic field emitted by the electromagnet. In various embodiments, a permanent magnet can include a material that can emit a magnetic field even when not subjected to an electric current. Various permanent magnets can be comprised of manufactured composites and/or rare-earth elements. Such manufactured composites can include ferrite magnets, which can include sintered composites of powdered iron oxides and barium and/or strontium carbonate, and alnico magnets, which can include a combination of aluminum, nickel, cobalt, and iron, for example. Rare-earth magnets can include magnets comprised of lanthanide elements, for example, and can include samarium-cobalt and neodymium-iron-boron magnets, for example. In any event, a permanent magnet and/or electromagnet can emit a magnetic field configured to attract or the magnetic implant positioned within the patient's body relative to the magnet to allow tissue engaged with the magnetic implant to be manipulated within the body cavity.

In various embodiments, a surgical instrument can include one ore more magnets which can be configured to emit magnetic fields having different intensities or magnetic moments. In at least one embodiment, a surgical instrument can include an electromagnet which can be configured to emit a first magnetic field having a first intensity, or magnetic moment, wherein the first magnetic field can be created by a flow of a first electrical current through the wire coils of the electromagnet. In at least one such embodiment, the first magnetic field can apply a first attractive force to a first magnetic implant engaged with the tissue as described above. In various embodiments, the flow of electrical current through the wires can be increased or decreased to correspondingly increase or decrease the intensity, or magnetic moment, of the magnetic field emitted by the electromagnet. In at least one embodiment, the electromagnet can be configured to emit a second magnetic field having a second intensity, or magnetic moment, at the same time as, or a different time than, when the electromagnet is emitting the first magnetic field. In at least one such embodiment, the second magnetic field can apply a second attractive force to a second magnetic implant engaged with the tissue. As a result of applying different magnetic fields of different intensities to various magnetic implants, the tissue engaged with the implants can be selectively manipulated within the body during a surgical procedure. In various embodiments, the magnetic implants can be configured such that they have different responses, or degrees of responses, to a magnetic field or moment. In at least one embodiment, for example, various magnetic implants can be comprised of different quantities or arrangements of magnetic material therein such that the magnetic implants can be more responsive, or less responsive, to a magnetic field or moment. Although the above-described example was explained in the context of an electromagnet, a surgical instrument can include permanent magnets which can be manipulated, such as being selectively positioned or moved relative to the magnetic implants to achieve the same or a similar result.

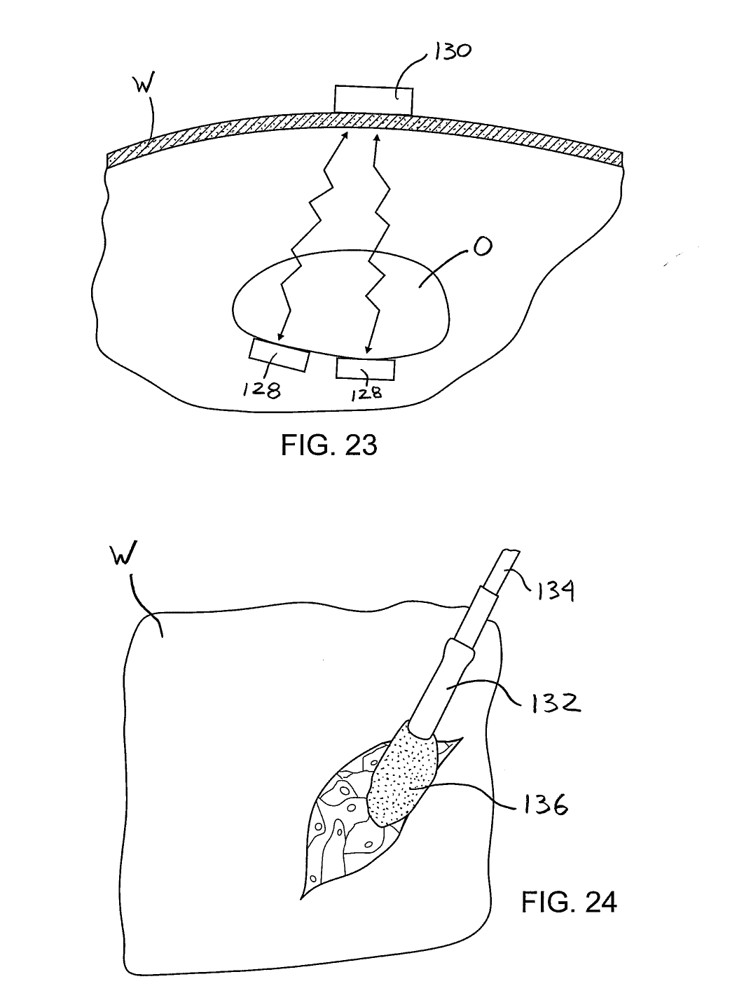

In various embodiments, referring to FIG. 23, one or more magnetic implants 128 can be positioned with respect to, or underneath, a first side of tissue within a patient's body, such as a first side of organ “O”, for example. In at least one embodiment, magnetic implant 128 can have any suitable shape, such as a pad, for example, which can be configured to contact the first side of organ O, for example. In various embodiments, implants 128 can be contoured such that the implants can be especially adapted to be positioned against certain organs, such as the liver, for example. In at least one embodiment, although not illustrated, implants 128 can include projections, or cleats, extending therefrom which can be configured to engage an organ such that the possibility of relative movement between the organ and implants 128 can be reduced. In various embodiments, a surgical instrument including a magnet, such as magnet 130, for example, which can be positioned relative to a second side of organ O. In at least one embodiment, similar to the above, magnet 130 can emit a magnetic field configured to attract magnetic implants 128 and move organ O toward magnet 130. Advantageously, such manipulation of organ O, for example, can occur without the use of sutures, clips, anchors, adhesives, and/or medical tape to attach the magnets to the tissue and, as a result, the possibility of damaging organ O can be reduced.

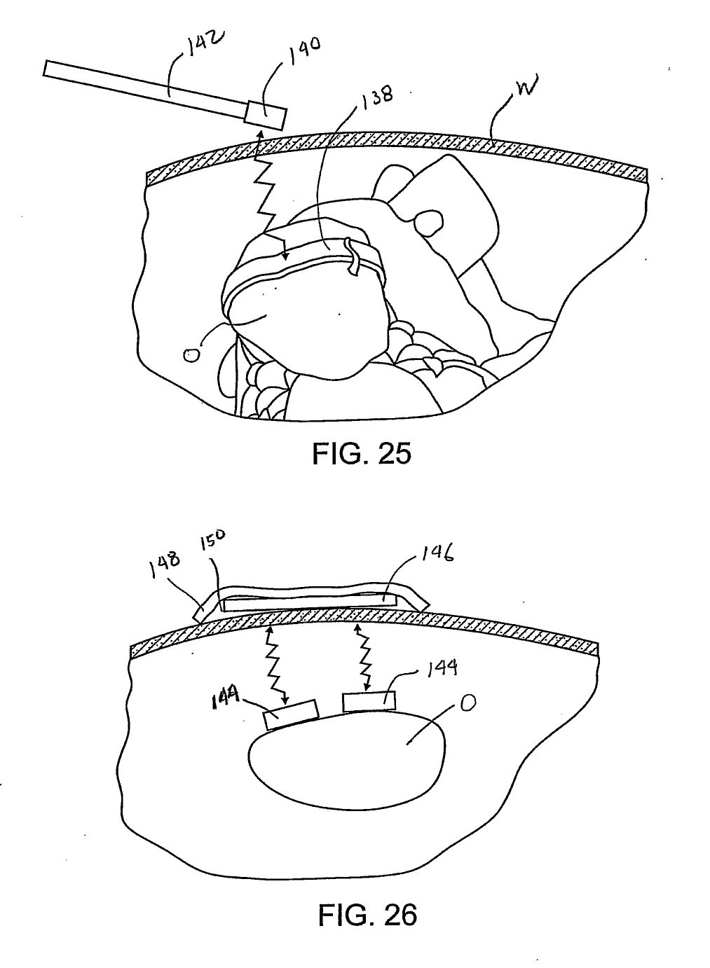

In various embodiments, referring to FIG. 25, a band, or strap, including a magnetic material can be configured to at least partially surround tissue, such as organ “O”, for example, such that the tissue can be manipulated within the body when a magnetic field is applied to the band. In at least one embodiment, band 138 can be positioned around, or slid over, organ O and, in various embodiments, band 138 can include a first end and a second end where at least one of the first and second ends can include a connector, such as a clip, snap, and/or Velcro® strip, for example, to connect the first end to the second end and secure the band around the organ. In at least one embodiment, band 138 can be at least partially comprised of a material such as, polyethylene, Teflon (PTFE), nylon, silicone, and/or latex, for example. In various embodiments, band 138 can include a tissue-contacting surface configured to grip the organ such that the organ can be inhibited from sliding out of the band when the organ is manipulated by the magnet on the surgical instrument 142. In at least one embodiment, the tissue-contacting surface can include at least one hook and/or gripping member extending therefrom, wherein the hook and/or gripping member can be configured to grip at least a portion of the organ when the band is positioned at least partially around the organ. In various embodiments, the band can include a cavity, or pocket, which can at least partially extend between the first and second ends of the band and which can be configured to receive at least one magnetic element therein comprised of a magnetic material. In at least one embodiment, such an element can include a solution having a plurality of magnetic particles, an implant comprised of a magnetic material, and/or a magnet, for example.

In use, an element comprised of a magnetic material can be introduced into band 138 before band 138 is inserted into a body cavity of a patient through a cannula, a trocar, and/or through a natural opening of the patient. Once in the body cavity, surgical instruments, such as graspers, for example, can be used to position and secure band 138 around organ O, for example. Once band 138 is positioned around organ O, surgical instrument 142, which can include magnet 140, for example, can be positioned relative to band 138 in order to motivate band 138, and organ O engaged therewith, via a magnetic field emitted by magnet 140. In use, as magnet 140 is moved in a first direction relative to wall W of the body cavity, organ O can be moved in the first direction and, likewise, as magnet 140 is moved in a second direction relative to wall W, organ O can be moved in the second direction, for example. In other various embodiments, a pouch including a magnetic material can be configured to at least partially surround tissue, such as organ “O”, for example, such that the tissue can be manipulated within the body when a magnetic field is applied to the pouch. In at least one embodiment, the pouch can define a cavity therein which can be configured to receive the tissue. The pouch can include at least one magnetic member which can be contained within, and/or positioned on, the pouch. In addition to or in lieu of the above, a pouch can include one or more pockets which can be configured to receive at least one magnetic member therein such that the magnetic members can be easily removed and/or replaced with different magnetic members.

In various embodiments, referring to FIG. 26, a surgical device kit can include at least one magnetic implant 144 which can, similar to the above, be attached to or engaged with tissue, such as organ “O”, for example, within and/or adjacent to a surgical site. The device kit can further include at least one magnet 146 and at least one blanket, or pad, 148 which can be positioned external to a patient's body. In various embodiments, pad 148 can include at least one cavity, or pocket, 150 which can be configured to receive magnet 146. In at least one embodiment, pad 148 can be flexible such that it can be laid over, and/or conform to, the patient wherein the magnets contained therein can be positioned adjacent to a side wall of the patient such that they can generate a magnetic field within the body cavity. In at least one such embodiment, as a result of the above, a surgeon, or other clinician, may not be required to hold a magnet, or a surgical instrument having a magnet, in place during a surgical procedure. In various embodiments, magnet 146 can comprise at least one permanent magnet. In addition to or in lieu of permanent magnets, magnet 146 can comprise at least one electromagnet. In at least one such embodiment, the electromagnets within pad 148 can be selectively actuated to create a desired magnetic field.

In various embodiments, referring to FIG. 24, a solution of magnetic particles can be introduced into, or otherwise applied to, an organ of a patient such that the organ can be manipulated relative to the patient's body by a magnet, such as magnet 132 attached to surgical instrument 134, for example. In at least one embodiment, the magnetic particles can be comprised of iron particles and/or other suitable magnetic particles having any suitable size and shape. In various embodiments, similar to the above, magnet 132 can emit a magnetic field which applies a force, such as an attractive force, to the magnetic particles in the solution such that the magnetic particles, and the organ associated therewith, can be manipulated relative to the body. In at least one embodiment, the solution can be applied to an external portion of the organ and/or can be injected into the organ through the use of a needle or other fluid injection device. In various embodiments, the solution can be inserted into pouch 136, referring to FIG. 24, where pouch 136 can be sealed and inserted into the organ via a trocar and/or a natural orifice of the patient. Thereafter, in at least one embodiment, a surgeon can utilize a magnet, such as magnet 132, for example, to remove the organ from the patient's body through an incision in the cavity wall. In various embodiments, pouch 136 can be at least partially comprised of a material such as silicone, polyester, and/or Gore-tex®, for example. In at least one embodiment, the solution inserted into the pouch can further include water, or any other fluid suitable for use within the body, to allow the pouch to be more easily handled.

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Preferably, the invention described herein will be processed before surgery. First, a new or used instrument is obtained and if necessary cleaned. The instrument can then be sterilized. In one sterilization technique, the instrument is placed in a closed and sealed container, such as a plastic or TYVEK bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility.

While this invention has been described as having exemplary designs, the present invention may be further modified within the spirit and scope of the disclosure. This application is therefore intended to cover any variations, uses, or adaptations of the invention using its general principles. Further, this application is intended to cover such departures from the present disclosure as come within known or customary practice in the art to which this invention pertains.

1. A surgical kit for use in manipulating tissue within a body of a patient, the kit comprising:
a first implant configured to be attached to a first portion of the tissue;
a second implant configured to be attached to a second portion of the tissue; and
a surgical instrument configured to be positioned relative to the body, wherein said surgical instrument is configured to emit a first magnetic field which can apply a first attractive force to said first implant to manipulate the first portion of the tissue within the body, wherein said surgical instrument is configured to emit a second magnetic field which can apply a second attractive force to said second implant to manipulate the second portion of the tissue within the body, and wherein said first magnetic field is different than said second magnetic field.

2. The surgical kit of claim 1, wherein said first magnetic field has a first intensity, wherein said second magnetic field has a second intensity, and wherein said first intensity is different than said second intensity.

3. The surgical kit of claim 1, wherein said first implant is comprised of a first magnetic material, wherein said second implant is comprised of a second magnetic material, and wherein said first magnetic material is different than said second magnetic material.

4. The surgical kit of claim 1, wherein said surgical instrument comprises a blanket having a first magnet configured to emit the first magnetic filed and a second magnet configured to emit the second magnetic field.

5. A surgical kit for use in manipulating tissue within the body of a patient, the kit comprising:
an insert configured to be positioned relative to a first side of the tissue, wherein said insert includes a magnetic material; and
a surgical instrument including a magnet, wherein said magnet is configured to be positioned relative to a second side of the tissue, and wherein said magnet is configured to emit a magnetic field configured to attract said magnetic material toward said magnet to lift the tissue within the body.

6. The surgical kit of claim 5, wherein said insert comprises a sheet of magnetic material.

7. The surgical kit of claim 5, wherein said insert is not configured to be attached to the tissue.

8. A surgical kit for use in manipulating tissue within the body of a patient, the kit comprising:
a band configured to at least partially surround the tissue, wherein said band includes a magnetic material; and
a surgical instrument including a magnet, wherein said magnet is configured to emit a magnetic field configured to motivate said magnetic material relative to said magnet to manipulate the tissue within the body.

9. The surgical kit of claim 8, wherein said band includes a pocket configured to receive said magnetic material.

10. The surgical kit of claim 8, wherein said band includes a first end and a second end, wherein said first end includes a first connector, wherein said second end includes a second connector, and wherein said first connector is configured to be operably engaged with said second connector to retain said band at least partially around the tissue.

11. The surgical kit of claim 8, wherein said band includes at least one hook configured to engage the tissue.

12. A surgical kit for use in manipulating tissue within the body of a patient, the kit comprising:
a pouch configured to at least partially surround the tissue, wherein said pouch includes a magnetic material; and
a surgical instrument including a magnet, wherein said magnet is configured to emit a magnetic field configured to motivate said magnetic material relative to said magnet to manipulate the tissue within the body.

13. The surgical kit of claim 12, wherein said pouch includes a pocket configured to receive said magnetic material.

14. The surgical kit of claim 12, wherein said pouch includes at least one hook configured to engage the tissue.

15. A surgical kit for use in manipulating tissue within the body of a patient, the kit comprising:
a magnetic implant configured to be engaged with the tissue; and
a blanket configured to be positioned external to the patient's body, wherein said blanket is configured to emit a magnetic field configured to motivate said magnetic implant relative to said blanket and manipulate the tissue within the body.

16. The surgical kit of claim 15, wherein said blanket includes a pocket configured to receive a magnet, and wherein said magnet is configured to emit the magnetic field.

17. The surgical kit of claim 15, wherein said blanket is configured to conform to at least a portion of the patient's body.

18-56. (canceled)
